(19) United States
(12) Patent Application Publication
(10) Pub. No.: US 2025/0302891 A1
(43) Pub. Date: Oct. 2, 2025

(54) MICROBIOME BIOMARKERS FOR IRRITABLE BOWEL DISEASE (IBD)

(71) Applicant: Gnubiotics Sciences SA, Epalinges (CH)

(72) Inventor: Adeyemi Adesokan, Epalinges (CH)

(73) Assignee: Gnubiotics Sciences SA, Epalinges (CH)

(21) Appl. No.: 19/096,704

(22) Filed: Mar. 31, 2025

Related U.S. Application Data
(60) Provisional application No. 63/572,791, filed on Apr. 1, 2024.

(51) Int. Cl. A61K 35/742; A61K 38/17; A61K 47/36; C12Q 1/04

(52) U.S. Cl. CPC A61K 35/742; A61K 38/1735; A61K 47/36; C12Q 1/04; G01N 2800/065; G01N 2800/50; G01N 2800/52

(57) ABSTRACT

Provided herein are methods for detecting inflammatory bowel disease (IBD) or risk of IBD in a canine based on the canine's gut microbiome. Also provided are methods for treating or preventing IBD in canines. Finally provided are probiotic compositions that are used in the methods of the present disclosure.

### CROSS-REFERENCE TO RELATED PATENT APPLICATION

This application claims the benefit under 35 U.S.C. § 119 (e) of U.S. Provisional Application No. 63/572,791, filed on Apr. 1, 2024, the entire contents of which are incorporated herein by reference.

### BACKGROUND

Inflammatory bowel disease (IBD) is a chronic condition in domestic dogs caused by inflammatory cells infiltrating the intestine lining, leading to allergic reactions, poor digestion, vomiting, diarrhea, poor appetite, and weight loss. The definitive diagnosis approach for IBD in dogs is mainly based on invasive tissue biopsies, which are obtained through surgical procedures. However, this approach is invasive, expensive and requires the services of a veterinary pathologist. Therefore, better methods for detecting and treating IBD are needed.

### SUMMARY OF THE DISCLOSURE

In one aspect, provided herein is a probiotic composition comprising, or alternatively consisting essentially of, or yet further consisting of at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques or Slackia faecicanis, and a pharmaceutically acceptable carrier or preservative.

In some embodiments, the probiotic composition further comprises, or alternatively consists essentially of, or yet further consists of at least one bacterial species selected from Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis or Romboutsia lituseburensis.

In some embodiments, the probiotic composition further comprises, or alternatively consists essentially of, or yet further consists of one or both of bacterial species Blautia hansenii and Clostridium perfringens.

In some embodiments, the probiotic composition further comprises, or alternatively consists essentially of, or yet further consists of at least one prebiotic selected from the group consisting of: fructo-oligosaccharides, short chain fructo-oligosaccharides, inulin, isomalto-oligosaccharides, pectins, galacto-oligosaccharides, arabinogalactan, xylo-oligosaccharides, chitosan-oligosaccharides, glucomannan, betaglucans, Konjac, guar, arabic, xanthan gums, modified and resistant starches, polydextrose, and D-tagatose.

Another aspect of the disclosure is directed to a method for identifying inflammatory bowel disease (IBD) or risk of IBD in a domesticated dog comprising, or alternatively consisting essentially of, or yet further consisting of: (a) measuring the levels of (i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, in a biological sample from the domesticated dog relative to pre-determined threshold expression level of the corresponding bacterial species; and (b) determining that the domesticated dog has IBD or at risk of IBD when the level of the bacterial species measured in step (a) (i) are lower than a pre-determined threshold level of the corresponding bacterial species, or when the level of the bacterial species measured in step (a) (ii) is higher than a pre-determined threshold level of the corresponding bacterial species.

In some embodiments, the biological sample is a fecal sample.

Another aspect of the disclosure is directed to a method for treating inflammatory bowel disease (IBD) or risk of IBD in a domesticated dog comprising, or alternatively consisting essentially of, or yet further consisting of: (a) receiving information as to the levels of (i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, in a biological sample from the domesticated dog relative to a pre-determined threshold level of the corresponding bacterial species; (b) determining that the domesticated dog has IBD or at risk of IBD when the level of the bacterial species measured in step (a) (i) is lower than a pre-determined threshold level of the corresponding bacterial species, or when the level of the bacterial species measured in step (a) (ii) is higher than a pre-determined threshold level of the corresponding bacterial species; and (c) treating the domesticated dog determined to have IBD or at risk of IBD in step (b), by administering to the domesticated dog a therapeutically effective amount of (1) a composition comprising, or alternatively consisting essentially of, or yet further consisting of an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (2) a composition comprising, or alternatively consisting essentially of, or yet further consisting of an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or (3) a combination of compositions of (1) or (2).

In some embodiments, the biological sample is a fecal sample.

In some embodiments, the agent that selectively increases the gut population of one or more bacterial species in (1) or that selectively decreases the gut population of one or more bacterial species in (2) comprises, or alternatively consists essentially of, or yet further consists of mucins.

In some embodiments, the agent comprises, or alternatively consists essentially of, or yet further consists of porcine mucins obtained from porcine gastrointestinal tract.

In some embodiments, the agent comprises, or alternatively consists essentially of, or yet further consists of less than 1% free glycans.

In some embodiments, the agent that selectively increases the gut population of one or more bacterial species in (1) comprises, or alternatively consists essentially of, or yet further consists of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

Another aspect of the disclosure is directed to a method for preventing inflammatory bowel disease (IBD) in a domesticated dog, comprising, or alternatively consisting essentially of, or yet further consisting of administering to the domesticated dog an effective amount of (1) a composition comprising, or alternatively consisting essentially of, or yet further consisting of an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (2) a composition comprising, or alternatively consisting essentially of, or yet further consisting of an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or (3) a combination of compositions of (1) or (2).

In some embodiments, the agent that selectively increases the gut population of one or more bacterial species in (1) or that selectively decreases the gut population of one or more bacterial species in (2) comprises, or alternatively consists essentially of, or yet further consists of mucins.

In some embodiments, the agent comprises, or alternatively consists essentially of, or yet further consists of porcine mucins obtained from porcine gastrointestinal tract.

In some embodiments, the agent comprises, or alternatively consists essentially of, or yet further consists of less than 1% free glycans.

In some embodiments, the agent that selectively increases the gut population of one or more bacterial species in (1) comprises, or alternatively consists essentially of, or yet further consists of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

Another aspect of the disclosure is directed to a method of treating or preventing inflammatory bowel disease (IBD) or risk of IBD in a domesticated dog in need thereof comprising, or alternatively consisting essentially of, or yet further consisting of administering to the domesticated dog an effective amount of (1) a composition comprising, or alternatively consisting essentially of, or yet further consisting of an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (2) a composition comprising, or alternatively consisting essentially of, or yet further consisting of an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or (3) a combination of compositions of (1) or (2), wherein a sample isolated from the domesticated dog in need thereof has levels of a measured bacterial species in step (i) are lower than a pre-determined threshold level of the corresponding bacterial species, or when the levels of the measured species in step (ii) are higher than a pre-determined threshold level of the corresponding bacterial species, and wherein the measured bacterial species comprise one or more of: (i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis.

In some embodiments, the agent that selectively increases the gut population of one or more bacterial species in (1) or that selectively decreases the gut population of one or more bacterial species in (2) comprises, or alternatively consists essentially of, or yet further consists of mucins.

In some embodiments, the agent comprises, or alternatively consists essentially of, or yet further consists of porcine mucins obtained from porcine gastrointestinal tract.

In some embodiments, the agent comprises, or alternatively consists essentially of, or yet further consists of less than 1% free glycans.

### BRIEF DESCRIPTION OF THE FIGURES

FIG. 1 provides information regarding domesticated dogs that were assessed for microbiome markers.

FIG. 2 shows Bacteroides plebeius, Clostridium saudiense, Fusobacterium varium, Holdemanella biformis, Phascolarctobacterium succinatutens, Prevotella copri, Romboutsia timonensis, Ruminococcus torques and Slackia faecicanis as potential biomarkers of good health in domesticated dogs. FIG. 2 shows that none of the IBD dogs have Bacteroides plebeius, Clostridium saudiense, Fusobacterium varium, Holdemanella biformis, Phascolarctobacterium succinatutens, Prevotella copri, Romboutsia timonensis, Ruminococcus torques and Slackia faecicanis in their gut, but the healthy dogs have those bacteria.

FIG. 3 demonstrates that an enrichment of a dog gut microbiome above the pre-determined threshold with any one or more of Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis and Tyzzerella nexilis can be used to identify dogs with IBD. FIG. 3 also shows that a reduction of a dog gut microbiome below the pre-determined threshold with any one or more of Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis and Romboutsia lituseburensis can be used to identify dogs with IBD.

FIG. 4 shows that, although Bacillus bogoriensis, Faecalimonas umbilicate, Ruminococcus gnavus, Blautia hansenii and Clostridium perfringens were identified in all healthy and IBD dogs, Bacillus bogoriensis, Faecalimonas umbilicate, Ruminococcus gnavus were clearly enriched whereas Blautia hansenii and Clostridium perfringens were reduced in IBD dogs compared to healthy controls.

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Techique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, agents, compositions and methods include the recited elements, but not exclude others. “Consisting essentially of” when used to define compounds, agents, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination and can be separately packaged with or without specific dosages. The active ingredients of the combination can be administered concurrently or sequentially.

An “effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific agent employed, bioavailability of the agent, the route of administration, the age of the animal and its body weight, general health, sex, the diet of the animal, the time of administration, the rate of excretion, the drug combination, and the severity of the particular disorder being treated and form of administration. In general, one will desire to administer an amount of the agent that is effective to achieve a serum level commensurate with the concentrations found to be effective in vivo. These considerations, as well as effective formulations and administration procedures are well known in the art and are described in standard textbooks.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the phrase “derived from” means isolated from, purified from, or engineered from, or any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “animal” refers to a canine. A “canine” is a mammal of the family Canidae, including the canids. Non-limiting examples include wolves, jackals, foxes, dogs, and coyotes. In some embodiments, the canine is a dog or a domesticated dog.

The term “subject,” “host,” “individual,” and “patient” are as used interchangeably herein to refer to animals, typically a canine. In some embodiments, a subject is a domesticated dog.

In one embodiment, the term “disease” or “disorder” as used herein refers to irritable bowel disease (aka. irritable bowel syndrome).

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of an oncolytic virus or a composition containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is administration to a tumor microenvironment. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more.

The term administration shall include without limitation, administration by oral, parenteral (e.g., intramuscular, intraperitoneal, intravenous, intravascular, intraperitoneal, intracerebroventricular (ICV), intrathecal, intracisternal injection or infusion, intracranial, ocular, intradermally, percutaneously, subcutaneous injection, or implant), intratumorally, by inhalation spray nasal, intratracheal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

An agent of the present disclosure can be administered for therapy by any suitable route of administration. It will also be appreciated that the optimal route will vary with the condition and age of the recipient, and the disease being treated.

Administration or treatment in “combination” refers to administering two agents such that their pharmacological effects are manifest at the same time. Combination does not require administration at the same time or substantially the same time, although combination can include such administrations.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “purified” does not require absolute purity; rather, it is intended as a relative term. Thus, for example, a glycoprotein, mucin, purified nucleic acid, peptide, protein, biological complexes, cell, virus or other active compound is one that is isolated in whole or in part from proteins or other contaminants. Generally, substantially purified glycoprotein, mucin, peptides, proteins, biological complexes, cell, virus or other active compounds for use within the disclosure comprise more than 80% of all macromolecular species present in a preparation prior to admixture or formulation of the glycoprotein, mucin, peptide, protein, biological complex, cell, virus or other active compound with a pharmaceutical carrier, excipient, buffer, absorption enhancing agent, stabilizer, preservative, adjuvant or other co-ingredient in a complete pharmaceutical formulation for therapeutic administration. More typically, the glycoprotein, mucin, peptide, protein, biological complex, cell, virus or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention.

The term “prebiotic” refers to food ingredients that are not readily digestible by endogenous host enzymes and confer beneficial effects on an organism that consumes them by selectively stimulating the growth and/or activity of a limited range of beneficial microorganisms that are associated with the intestinal tract.

The term “probiotic” refers to one or more live microorganisms that confer beneficial effects on a host organism.

A “pre-determined threshold” as used herein refers to a quantitative threshold value or range for a selected bacterial species. The threshold value provides a point or range at which a bacterial species acts as a biomarker for obesity or leanness. A pre-determined threshold level may be a fixed value or a value range for each individual bacterial species (e.g., unique pre-determined threshold values for Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens). In some aspects, a pre-determined threshold value may be calculated by measuring the level or DNA copy number presence of a specific bacterial species from a control sample obtained from a canine, e.g., domesticated dog (e.g., a domesticated dog that does not qualify as having IBD). In some embodiments, a pre-determined threshold value may be calculated by measuring the level or DNA copy number presence of a specific bacterial species from a control sample obtained from the average of a group (e.g., 5 or more, 10 or more, 20 or more, etc.) of domesticated dogs (e.g., domesticated dogs that do not qualify as having IBD). In some aspects, the bacterial species are characterized as being between 15% and 95%, e.g., between 20% and 80%, between 25% and 75%, between 30% and 70%, or between 40% and 60% above or below a pre-determined threshold for the selected bacterial species.

As used herein, a “reduced level” of expression or activity is a level of expression or activity that is detectably lower than a pre-determined threshold or reference level. In some embodiments, a reduced level of expression or activity is between 10% and 95% of a pre-determined threshold, although lesser and greater reductions are contemplated in some embodiments. In some embodiments expression or activity is reduced by about 75%, about 80%, about 85%, about 90%, about 95%, about 98%, or about 100%. In some embodiments a reduced level of expression or activity is between 0% and 5% of a pre-determined threshold. In some embodiments a reduced level of expression or activity is between 5% and 15% of a pre-determined threshold. In some embodiments a reduced level of expression or activity is between 15% and 95%, e.g., between 20% and 80%, between 25% and 75%, between 30% and 70%, or between 40% and 60% of a pre-determined threshold. In some embodiments a reduced level of expression or activity is between 25% and 50% or between 50% and 75% of a pre-determined threshold. In some embodiments a reduced level of expression or activity is about 1%, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 97%, about 99% or about 100% of a pre-determined threshold (but less than the pre-determined threshold).

As used herein, an “increased level” of expression or activity is a level of expression or activity that is detectably higher than a pre-determined threshold. In some embodiments, an increased level of expression or activity is between 10% and 100% above a pre-determined threshold, although lesser and greater increases are contemplated in some embodiments. In some embodiments an increased level of expression or activity is between 15% and 95%, e.g., between 20% and 80%, between 25% and 75%, between 30% and 70%, or between 40% and 60% above a pre-determined threshold. In some embodiments an increased level of expression or activity is between 25% and 50% or between 50% and 75% above a pre-determined threshold. In some embodiments an increased level of expression or activity is about 10%, about 20%, about 25%, about 30%, about 35%, about 40%, about 45%, about 50%, about 55%, about 60%, about 65%, about 70%, about 75%, about 80%, about 85%, about 90%, about 95%, about 97%, about 99% or about 100% above a pre-determined threshold. In some embodiments an increased level of expression or activity is increased over a pre-determined threshold by a factor of at least 2-fold, at least 3-fold, at least 4-fold, at least 5-fold, at least 6-fold, at least 7-fold, at least 8-fold, at least 9-fold, or at least 10-fold.

The term “mucin” encompasses any natural source of mucin from which glycans and glycopeptides can be extracted, suitable for mammalian nutrition or pharmaceutical use. Typical sources of mucins are extracts from gastrointestinal tract, in particular from porcine sources or from bovine sources. Commercial sources for gastrointestinal tract mucins include Biofac A/S (Kastrup, Denmark), Zhongshi Duqing (Heze, China), Shenzhen Taier Biotechnology Co., LTD (Shenzhen, China), and Dongying Tiandong Pharmaceutical Co. (Shandong, China). In some embodiments, the gastrointestinal tract mucins are from porcine gastric mucus.

The term “glycan” as used herein refers to an oligosaccharide. The term “free glycan” is synonymous with the term “free oligosaccharide,” and refers to a glycan that is not attached to a peptide/protein.

The term “glycoprotein” refers to proteins linked to oligosaccharides, e.g., proteins either N-linked or O-linked to oligosaccharides, and having a molecular weight of more than about 5 KDa.

The term “glycopeptide” refers to peptides linked to oligosaccharides, e.g. peptides either N-linked or O-linked to oligosaccharides, and having a molecular weight of less than about 5 KDa. Methods of determining molecular weight of glycopeptides and glycoproteins are known in the art and are not limited. In some embodiments, the molecular weight of glycopeptides and glycoproteins are determined by size exclusion chromatography.

In some embodiments, peptides are defined as having a molecular weight of less than about 5 KDa. In some embodiments, the term peptides include glycopeptides. In some embodiments, proteins are defined as having a molecular weight of more than about 5 KDa. In some embodiments, the term proteins include glycoproteins.

### MODES FOR CARRYING OUT THE DISCLOSURE

Aspects of the disclosure relate to identifying specific bacterial species that are used as biomarkers for irritable bowel disease (IBD). The identified biomarkers are used for diagnostic and therapeutic purposes. The disclosure also provides probiotic compositions that are used to treat canines and domesticated dogs suffering from IBD.

Generally disclosed herein are methods for diagnosing a disease (e.g., IBD) based on the levels of one or more biomarkers. Methods of diagnosing or identifying a canine such as a domesticated dog for IBD or a predisposition to IBD are disclosed herein. Also disclosed herein are methods of identifying a canine such as a domesticated dog for treatment with an anti-IBD agent. In some embodiments the methods comprise determining if a canine such as a domesticated dog or a sample isolated from the canine such as a domesticated dog has one or more bacterial species above a pre-determined threshold. In some embodiments the methods comprise determining if a canine such as a domesticated dog has one or more bacterial species below a pre-determined threshold.

### Probiotic Compositions

The probiotic compositions described herein are used to treat or prevent IBD in canine such as a domesticated dog in need thereof. Compositions for use in these treatments are also provided herein.

In one aspect, provided is a probiotic composition comprising, or consisting of or further consisting of at least three (e.g., at least three, at least four, at least five, at least six, at least seven, at least eight or all nine) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques or Slackia faecicanis, and a carrier such as a pharmaceutically acceptable carrier.

In some embodiments, the probiotic composition comprises, or alternatively consists essentially of, or yet further consists of at least one (e.g., at least one, at least two, at least three, at least four, at least five, at least six, at least seven, at least eight, at least nine, at least ten, at least eleven, at least twelve, at least thirteen or all fourteen) bacterial species selected from Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis or Romboutsia lituseburensis.

In some embodiments, the probiotic composition comprises, or alternatively consists essentially of, or yet further consists of one or both of bacterial species Blautia hansenii and/or Clostridium perfringens.

In some embodiments, the probiotic composition further comprises, or alternatively consists essentially of, or yet further consists of at least one prebiotic selected from the group consisting of: fructo-oligosaccharides, short chain fructo-oligosaccharides, inulin, isomalto-oligosaccharides, pectins, galacto-oligosaccharides, arabinogalactan, xylo-oligosaccharides, chitosan-oligosaccharides, glucomannan, betaglucans, Konjac, guar, arabic, xanthan gums, modified and resistant starches, polydextrose, and D-tagatose.

The probiotic composition can be formulated to provide between 1 million and 100 billion viable bacterial cells per serving, such as between 10 million and 10 billion viable bacterial cells per serving, or between 500 million and 2 billion viable bacterial cells per serving.

In some embodiments, the probiotic composition is in the form of a powder, and can be used in the form of preparations for oral administration such as tablets, hard capsules or soft capsules, liquids, or suspensions. In some embodiments, the probiotic composition further comprises excipients, binding agents, disintegrating agents, lubricants, solubilizing agents, suspending agents, or preservatives.

### Methods for Identifying Inflammatory Bowel Disease (IBD) or Risk of IBD

Another aspect of the disclosure is directed to a method for identifying inflammatory bowel disease (IBD) or risk of IBD in a canine such as a domesticated dog the method comprising, or alternatively consisting essentially of, or yet further consisting of: (a) measuring the levels of (i) at least three (e.g., at least 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (ii) at least one (e.g., at least 1, 2, 3, 4, 5, 6, 7, 8, 9, 10, 11, 12, 13, 14, 15, 16, 17, 18, 19, 20, or 21) bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, in a biological sample isolated from the canine such as a domesticated dog relative to pre-determined threshold expression level of the corresponding bacterial species; and (b) determining that the canine such as a domesticated dog has IBD or at risk of IBD when the level of the bacterial species measured in step (a) (i) are lower than a pre-determined threshold level of the corresponding bacterial species, or when the level of the bacterial species measured in step (a) (ii) is higher than a pre-determined threshold level of the corresponding bacterial species.

In some embodiments, the biological sample is a fecal sample.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### Methods for Treating Inflammatory Bowel Disease (IBD) or Risk of IBD

Another aspect of the disclosure is directed to a method for treating a canine such as a domesticated dog having inflammatory bowel disease (IBD) or at risk of IBD comprising, or consisting essentially of, or yet further consisting of administering to the canine such as a domesticated dog a probiotic composition of the present disclosure.

Another aspect of the disclosure is directed to a method for treating a canine such as a domesticated dog having inflammatory bowel disease (IBD) or at risk of IBD comprising, or alternatively consisting essentially of, or yet further consisting of: (a) receiving information as to the levels of (i) at least three (e.g., at least 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, in a biological sample from the canine such as a domesticated dog relative to a pre-determined threshold level of the corresponding bacterial species; (b) determining that the canine such as a domesticated dog has IBD or at risk of IBD when the level of the bacterial species measured in step (a) (i) is lower than a pre-determined threshold level of the corresponding bacterial species, or when the level of the bacterial species measured in step (a) (ii) is higher than a pre-determined threshold level of the corresponding bacterial species; and (c) treating the canine such as a domesticated dog determined to have IBD or at risk of IBD in step (b), by administering to the canine such as a domesticated dog a therapeutically effective amount of (1) a composition comprising, or alternatively consisting essentially of, or yet further consisting of an agent that selectively increases the gut population of one or more (e.g., 1, 2, 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (2) a composition comprising, or alternatively consisting essentially of, or yet further consisting of an agent that selectively decreases the gut population of one or more (e.g., 1, 2, 3, 4, 5, 6, 7, 8, 9, 10, 11, 12, 13, 14, 15, 16, 17, 18, 19, 20, or 21) bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or (3) a combination of compositions of (1) or (2).

In some embodiments, the biological sample is a fecal sample.

In some embodiments, the agent that selectively increases the gut population of one or more bacterial species in (1) or that selectively decreases the gut population of one or more bacterial species in (2) comprises, or alternatively consists essentially of, or yet further consists of mucins.

In some embodiments, the agent comprises, or alternatively consists essentially of, or yet further consists of porcine mucins. In some embodiments, the porcine mucins are obtained from porcine gastrointestinal tract.

In some embodiments, the agent comprises, or alternatively consists essentially of, or yet further consists of less than 1% free glycans (e.g., 0.9%, 0.8%, 0.7%, 0.6%, 0.5%, 0.4%, 0.3%, 0.2%, 0.1% or less) by weight. In some embodiments, the agent comprises substantially no free glycans.

In some embodiments, the agent comprises a plurality of glycopeptides derived from porcine gastrointestinal mucins and at least 90% of the plurality of glycopeptides in the agent are less than 2 KDa in size. In some embodiments, the glycopeptides in the plurality of glycopeptides are less than 15 amino acids. In some embodiments, the glycopeptides in the plurality of glycopeptides are between 8 and 15 amino acids in length. In some embodiments, the plurality of glycopeptides is obtained without subjecting the porcine gastrointestinal mucins to conditions or reagents that cause release of oligosaccharides from glycopeptides. In some embodiments, the plurality of glycopeptides of the present disclosure are prepared by a method that retains oligosaccharides on the glycopeptides while cleaving the peptide backbone into short peptides of between 8-15 amino acids.

In some embodiments, the plurality of glycopeptides comprises, consists essentially of or consists of the following oligosaccharide structures: Fucα1-2Galβ1-3GalNAc, Galβ1-3 (GlcNAcβ1-6) GalNAc, Galβ1-3 (6SGlcNAcβ1-6) GalNAc, Galβ1-3 (NeuAcα2-6) GalNAc, NeuAcα2-3Galβ1-3GalNAc, Galβ1-3 (NeuGcα2-6) GalNAc, NeuGcα2-3Galβ1-3GalNAc, Galβ1-3 (Galβ1-4GlcNAcβ1-6) GalNAc, Fucα1-2Galβ1-3 (6S-GlcNAcβ1-6) GalNAc, Galβ1-3 (Galα1-3Galβ1-4GlcNAcβ1-6) GalNAc, and NeuAcα2-3Galβ1-3 [(6S) GlcNAcβ1-6] GalNAc.

In some embodiments, the agent comprises between about 0.5-1% Fucα1-2Galβ1-3GalNAc containing glycopeptides, between about 0.5-1% Galβ1-3 (GlcNAcβ1-6) GalNAc containing glycopeptides, between about 3-5% Galβ1-3 (6SGlcNAcβ1-6) GalNAc containing glycopeptides, between about 1-3% Galβ1-3 (NeuAcα2-6) GalNAc, between about 1-3% NeuAcα2-3Galβ1-3GalNAc containing glycopeptides, between about 1-3% Galβ1-3 (NeuGcα2-6) GalNAc containing glycopeptides, between about 1-3% NeuGcα2-3Galβ1-3GalNAc containing glycopeptides, between about 0.5-1% Galβ1-3 (Galβ1-4GlcNAcβ1-6) GalNAc containing glycopeptides, between about 3-5% Fucα1-2Galβ1-3 (6S-GlcNAcβ1-6) GalNAc containing glycopeptides, between about 0.5-1% Galβ1-3 (Galα1-3Galβ1-4GlcNAcβ1-6) GalNAc containing glycopeptides, and between about 0.5-1% NeuAcα2-3Galβ1-3 [(6S) GlcNAcβ1-6] GalNAc containing glycopeptides by weight of the total composition.

In some embodiments, the agent contains less than 5%, less than 4%, less than 3%, less than 2% or less than 1% of insoluble particles having a diameter greater than about or 7 μm. In some embodiments, the agent contains substantially no insoluble particles having a diameter greater than 7 μm. In some embodiments, the diameter of at least 95%, 96%, 97%, 98%, 99% or more of insoluble particles in the agent is less than or equal to 7 μm (e.g., 7, 6, 5, 4, 3, 2, 1 μm or smaller).

In some embodiments, the agent that selectively increases the gut population of one or more bacterial species in (1) comprises, or alternatively consists essentially of, or yet further consists of one or more (e.g., 1, 2, 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

Another aspect of the disclosure is directed to a method for preventing inflammatory bowel disease (IBD) in a canine such as a domesticated dog, comprising, or alternatively consisting essentially of, or yet further consisting of administering to the canine such as a domesticated dog an effective amount of: (1) a composition comprising, or alternatively consisting essentially of, or yet further consisting of an agent that selectively increases the gut population of one or more (e.g., 1, 2, 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (2) a composition comprising, or alternatively consisting essentially of, or yet further consisting of an agent that selectively decreases the gut population of one or more (e.g., 1, 2, 3, 4, 5, 6, 7, 8, 9, 10, 11, 12, 13, 14, 15, 16, 17, 18, 19, 20, or 21) bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or (3) a combination of compositions of (1) or (2).

In some embodiments, the agent that selectively increases the gut population of one or more bacterial species in (1) or that selectively decreases the gut population of one or more bacterial species in (2) comprises, or alternatively consists essentially of, or yet further consists of mucins. In some embodiments, the agent comprises, or alternatively consists essentially of, or yet further consists of porcine mucins.

In some embodiments, the agent comprises, or alternatively consists essentially of, or yet further consists of less than 1% free glycans (e.g., 0.9%, 0.8%, 0.7%, 0.6%, 0.5%, 0.4%, 0.3%, 0.2%, 0.1% or less) by weight. In some embodiments, the agent comprises substantially no free glycans.

In some embodiments, the agent comprises a plurality of glycopeptides derived from porcine gastrointestinal mucins and at least 90% of the plurality of glycopeptides in the agent are less than 2 KDa in size. In some embodiments, the glycopeptides in the plurality of glycopeptides are less than 15 amino acids. In some embodiments, the glycopeptides in the plurality of glycopeptides are between 8 and 15 amino acids in length. In some embodiments, the plurality of glycopeptides is obtained without subjecting the porcine gastrointestinal mucins to conditions or reagents that cause release of oligosaccharides from glycopeptides.

In some embodiments, the plurality of glycopeptides comprises the following oligosaccharide structures: Fucα1-2Galβ1-3GalNAc, Galβ1-3 (GlcNAcβ1-6) GalNAc, Galβ1-3 (6SGlcNAcβ1-6) GalNAc, Galβ1-3 (NeuAcα2-6) GalNAc, NeuAcα2-3Galβ1-3GalNAc, Galβ1-3 (NeuGcα2-6) GalNAc, NeuGcα2-3Galβ1-3GalNAc, Galβ1-3 (Galβ1-4GlcNAcβ1-6) GalNAc, Fucα1-2Galβ1-3 (6S-GlcNAcβ1-6) GalNAc, Galβ1-3 (Galα1-3Galβ1-4GlcNAcβ1-6) GalNAc, and NeuAcα2-3Galβ1-3 [(6S) GlcNAcβ1-6] GalNAc.

In some embodiments, the agent comprises between about 0.5-1% Fucα1-2Galβ1-3GalNAc containing glycopeptides, between about 0.5-1% Galβ1-3 (GlcNAcβ1-6) GalNAc containing glycopeptides, between about 3-5% Galβ1-3 (6SGlcNAcβ1-6) GalNAc containing glycopeptides, between about 1-3% Galβ1-3 (NeuAcα2-6) GalNAc, between about 1-3% NeuAcα2-3Galβ1-3GalNAc containing glycopeptides, between about 1-3% Galβ1-3 (NeuGcα2-6) GalNAc containing glycopeptides, between about 1-3% NeuGcα2-3Galβ1-3GalNAc containing glycopeptides, between about 0.5-1% Galβ1-3 (Galβ1-4GlcNAcβ1-6) GalNAc containing glycopeptides, between about 3-5% Fucα1-2Galβ1-3 (6S-GlcNAcβ1-6) GalNAc containing glycopeptides, between about 0.5-1% Galβ1-3 (Galα1-3Galβ1-4GlcNAcβ1-6) GalNAc containing glycopeptides, and between about 0.5-1% NeuAcα2-3Galβ1-3 [(6S) GlcNAcβ1-6]GalNAc containing glycopeptides by weight of the total composition.

In some embodiments, the agent contains less than 5%, less than 4%, less than 3%, less than 2% or less than 1% of insoluble particles having a diameter greater than 7 μm. In some embodiments, the agent contains substantially no insoluble particles having a diameter greater than 7 μm. In some embodiments, the diameter of at least 95%, 96%, 97%, 98%, 99% or more of insoluble particles in the agent is less than or equal to 7 μm (e.g., 7, 6, 5, 4, 3, 2, 1 μm or smaller).

In some embodiments, the agent that selectively increases the gut population of one or more bacterial species in (1) comprises, or alternatively consists essentially of, or yet further consists of one or more (e.g., 1, 2, 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

Another aspect of the disclosure is directed to a method of treating or preventing inflammatory bowel disease (IBD) or risk of IBD in a canine such as a domesticated dog in need thereof comprising, or alternatively consisting essentially of, or yet further consisting of administering to the canine such as a domesticated dog an effective amount of (1) a composition comprising, or alternatively consisting essentially of, or yet further consisting of an agent that selectively increases the gut population of one or more (e.g., 1, 2, 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (2) a composition comprising, or alternatively consisting essentially of, or yet further consisting of an agent that selectively decreases the gut population of one or more (e.g., 1, 2, 3, 4, 5, 6, 7, 8, 9, 10, 11, 12, 13, 14, 15, 16, 17, 18, 19, 20, or all 21) bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or (3) a combination of compositions of (1) or (2), wherein a sample isolated from the canine such as a domesticated dog in need thereof has levels of a measured bacterial species in step (i) are lower than a pre-determined threshold level of the corresponding bacterial species, or when the levels of the measured species in step (ii) are higher than a pre-determined threshold level of the corresponding bacterial species, and wherein the measured bacterial species comprise one or more of: (i) at least three (e.g., at least 3, 4, 5, 6, 7, 8, 9, 10, 11, 12, 13, 14, 15, 16, 17, 18, 19, 20, 21, 22, 23, 24 or all 25) bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or (ii) at least one (e.g., at least 1, 2, 3, 4, 5, 6, 7, 8, 9, 10, 11, 12, 13, 14, 15, 16, 17, 18, 19, 20, or 21) bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis.

In some embodiments, the agent that selectively increases the gut population of one or more bacterial species in (1) or that selectively decreases the gut population of one or more bacterial species in (2) comprises, or alternatively consists essentially of, or yet further consists of mucins. In some embodiments, the agent comprises, or alternatively consists essentially of, or yet further consists of porcine mucins.

In some embodiments, the agent comprises, or alternatively consists essentially of, or yet further consists of less than 1% free glycans (e.g., 0.9%, 0.8%, 0.7%, 0.6%, 0.5%, 0.4%, 0.3%, 0.2%, 0.1% or less) by weight. In some embodiments, the agent comprises substantially no free glycans.

In some embodiments, the agent comprises a plurality of glycopeptides derived from porcine gastrointestinal mucins and at least 90% of the plurality of glycopeptides in the agent are less than 2 KDa in size. In some embodiments, the glycopeptides in the plurality of glycopeptides are less than 15 amino acids. In some embodiments, the glycopeptides in the plurality of glycopeptides are between 8 and 15 amino acids in length. In some embodiments, the plurality of glycopeptides is obtained without subjecting the porcine gastrointestinal mucins to conditions or reagents that cause release of oligosaccharides from glycopeptides.

In some embodiments, the plurality of glycopeptides comprises the following oligosaccharide structures: Fucα1-2Galβ1-3GalNAc, Galβ1-3 (GlcNAcβ1-6) GalNAc, Galβ1-3 (6SGlcNAcβ1-6) GalNAc, Galβ1-3 (NeuAcα2-6) GalNAc, NeuAcα2-3Galβ1-3GalNAc, Galβ1-3 (NeuGcα2-6) GalNAc, NeuGcα2-3Galβ1-3GalNAc, Galβ1-3 (Galβ1-4GlcNAcβ1-6) GalNAc, Fucα1-2Galβ1-3 (6S-GlcNAcβ1-6) GalNAc, Galβ1-3 (Galα1-3Galβ1-4GlcNAcβ1-6) GalNAc, and NeuAcα2-3Galβ1-3 [(6S) GlcNAcβ1-6]GalNAc.

In some embodiments, the agent comprises between about 0.5-1% Fucα1-2Galβ1-3GalNAc containing glycopeptides, between about 0.5-1% Galβ1-3 (GlcNAcβ1-6) GalNAc containing glycopeptides, between about 3-5% Galβ1-3 (6SGlcNAcβ1-6) GalNAc containing glycopeptides, between about 1-3% Galβ1-3 (NeuAcα2-6) GalNAc, between about 1-3% NeuAcα2-3Galβ1-3GalNAc containing glycopeptides, between about 1-3% Galβ1-3 (NeuGcα2-6) GalNAc containing glycopeptides, between about 1-3% NeuGcα2-3Galβ1-3GalNAc containing glycopeptides, between about 0.5-1% Galβ1-3 (Galβ1-4GlcNAcβ1-6) GalNAc containing glycopeptides, between about 3-5% Fucα1-2Galβ1-3 (6S-GlcNAcβ1-6) GalNAc containing glycopeptides, between about 0.5-1% Galβ1-3 (Galα1-3Galβ1-4GlcNAcβ1-6) GalNAc containing glycopeptides, and between about 0.5-1% NeuAcα2-3Galβ1-3 [(6S) GlcNAcβ1-6]GalNAc containing glycopeptides by weight of the total composition.

In some embodiments, the agent contains less than 5%, less than 4%, less than 3%, less than 2% or less than 1% of insoluble particles having a diameter greater than 7 μm. In some embodiments, the agent contains substantially no insoluble particles having a diameter greater than 7 μm. In some embodiments, the diameter of at least 95%, 96%, 97%, 98%, 99% or more of insoluble particles in the agent is less than or equal to 7 μm (e.g., 7, 6, 5, 4, 3, 2, 1 μm or smaller).

### Experimental Methods

Gut microbiome plays a crucial role in maintaining the health of the gastrointestinal tract and the immune system of dogs. Alterations in the composition and diversity of the gut microbiome may lead to an imbalance between beneficial and harmful bacteria in the gut, which can contribute to inflammation and disease.

### Methods

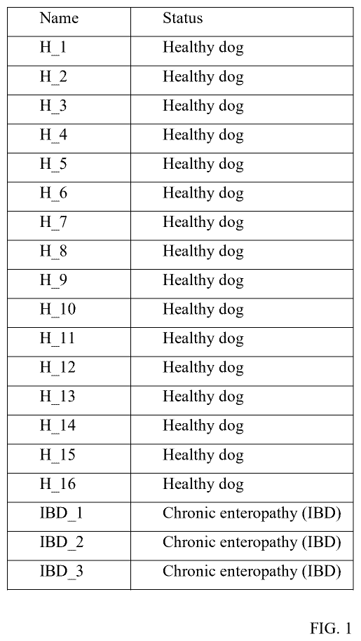

Using a novel high-resolution quantitative sequencing method, the presence and abundance of bacterial species in the gut of dogs were analyzed. DNA from the feces of clinically diagnosed 16 healthy and 3 IBD dogs, was sequenced (FIG. 1). For bioinformatics analysis, BLAST (see blast.ncbi.nlm.nih.gov/Blast.cgi) was used as the alignment tool run under default parameters, comparing each of the query datasets to each of the sample libraries, and setting parameters for optimal, tight alignments favoring identity matching versus homology matching. Potential gut microbiome biomarkers were validated with quantitative polymerase chain reaction (qPCR) using species-specific primers and SYBR Green-based detection method.

### Results

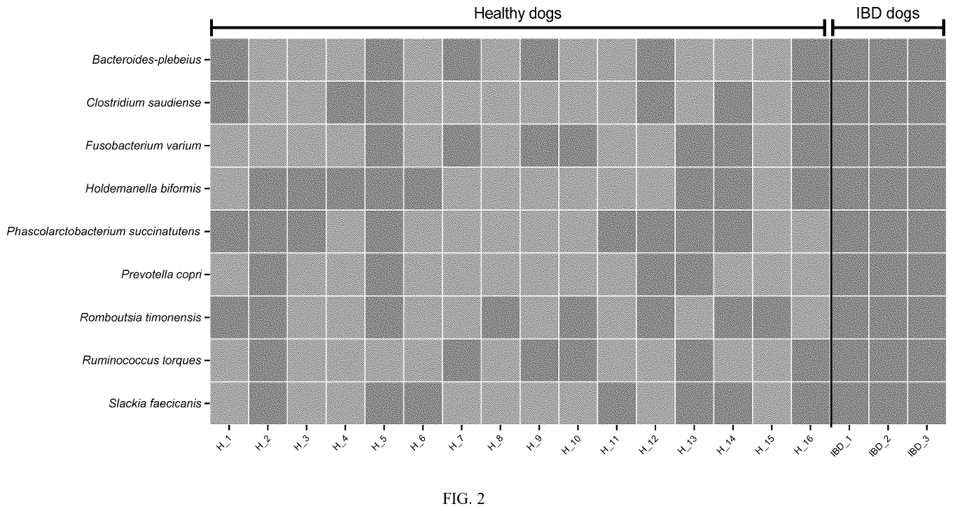

Among the 233 bacterial species identified, nine exhibited prevalence in over 50% of the healthy dogs, while these species remained absent in all IBD dogs (FIG. 2). These are Bacteroides plebeius, Clostridium saudiense, Fusobacterium varium, Holdemanella biformis, Phascolarctobacterium succinatutens, Prevotella copri, Romboutsia timonensis, Ruminococcus torques and Slackia faecicanis. These bacterial species were noted as potential biomarkers for good health in dogs.

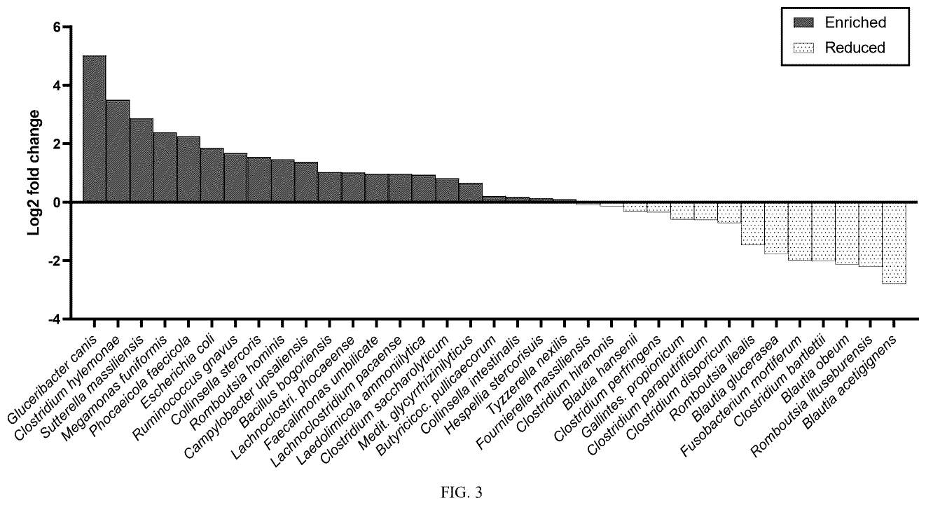

Additionally, 21 bacteria species were enriched while 14 species were decreased in at least 50% of the IBD dogs compared to healthy controls. The bacteria that were enriched in at least 50% of the IBD dogs are Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis and Tyzzerella nexilis. Those reduced in at least 50% of the IBD dogs are Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis and Romboutsia lituseburensis (FIG. 3).

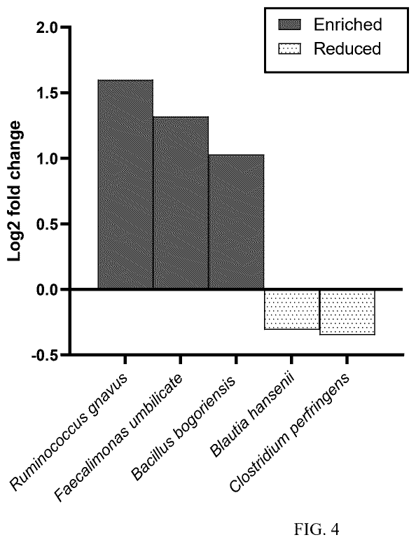

Five bacterial species were consistently present in all 19 stool samples from the dogs tested but displayed differential abundance in dogs with IBD, suggesting their potential as biomarkers of IBD. Bacillus bogoriensis, Faecalimonas umbilicate and Ruminococcus gnavus were enriched (log₂fold change>1.0) whereas Blautia hansenii and Clostridium perfringens were reduced (log₂fold change<1.0) in all IBD dogs compared to healthy controls (FIG. 4).

These results represent the first ever species-level analysis of microbiome markers of IBD in domesticated dogs and provide the basis for the diagnosis and treatment of this canine disease.

### ADDITIONAL EMBODIMENTS

Embodiment 1. A probiotic composition comprising at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques or Slackia faecicanis, and a pharmaceutically acceptable carrier.

Embodiment 2. The probiotic composition of embodiment 1, further comprising at least one bacterial species selected from Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis or Romboutsia lituseburensis.

Embodiment 3. The probiotic composition of embodiment 1 or embodiment 2, further comprising one or both of bacterial species Blautia hansenii and Clostridium perfringens.

Embodiment 4. The probiotic composition of any one of embodiments 1-3, further comprising at least one prebiotic selected from: fructo-oligosaccharides, short chain fructo-oligosaccharides, inulin, isomalto-oligosaccharides, pectins, galacto-oligosaccharides, arabinogalactan, xylo-oligosaccharides, chitosan-oligosaccharides, glucomannan, beta-glucans, Konjac, guar, arabic, xanthan gums, modified and resistant starches, polydextrose, or D-tagatose.

Embodiment 5. A method for identifying inflammatory bowel disease (IBD) or risk of IBD in a canine comprising:
(a) measuring the levels of
(i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis,
in a biological sample from the canine relative to pre-determined threshold expression level of the corresponding bacterial species;
and
(b) determining that the canine has IBD or at risk of IBD when the level of the bacterial species measured in step (a) (i) are lower than a pre-determined threshold level of the corresponding bacterial species, or when the level of the bacterial species measured in step (a) (ii) is higher than a pre-determined threshold level of the corresponding bacterial species.

Embodiment 6. The method of embodiment 5, wherein the biological sample is a fecal sample.

Embodiment 7. A method for treating inflammatory bowel disease (IBD) or risk of IBD in a canine comprising:
(a) receiving information as to the levels of
(i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis,
in a biological sample from the canine relative to a pre-determined threshold level of the corresponding bacterial species;
(b) determining that the canine has IBD or at risk of IBD when the level of the bacterial species measured in step (a) (i) is lower than a pre-determined threshold level of the corresponding bacterial species, or when the level of the bacterial species measured in step (a) (ii) is higher than a pre-determined threshold level of the corresponding bacterial species; and
(c) treating the canine determined to have IBD or at risk of IBD in step (b), by administering to the canine a therapeutically effective amount of
(1) a composition comprising an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(2) a composition comprising an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or
(3) a combination of compositions of (1) or (2).

Embodiment 8. The method of embodiment 7, wherein the biological sample is a fecal sample.

Embodiment 9. The method of embodiment 7 or embodiment 8, wherein the agent that selectively increases the gut population of one or more bacterial species in (1) or that selectively decreases the gut population of one or more bacterial species in (2) comprises mucins.

Embodiment 10. The method of any one of embodiments 7-9, wherein the agent comprises porcine mucins obtained from porcine gastrointestinal tract.

Embodiment 11. The method of any one of embodiments 7-10, wherein the agent comprises less than 1% free glycans.

Embodiment 12. The method of any one of embodiments 7-8, wherein the agent that selectively increases the gut population of one or more bacterial species in (1) comprises one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

Embodiment 13. A method for preventing inflammatory bowel disease (IBD) in a canine, comprising administering to the canine an effective amount of
(1) a composition comprising an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(2) a composition comprising an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or
(3) a combination of compositions of (1) or (2).

Embodiment 14. The method of embodiment 13, wherein the agent that selectively increases the gut population of one or more bacterial species in (1) or that selectively decreases the gut population of one or more bacterial species in (2) comprises mucins.

Embodiment 15. The method of embodiment 13 or embodiment 14, wherein the agent comprises porcine mucins obtained from porcine gastrointestinal tract.

Embodiment 16. The method of any one of embodiments 13-15, wherein the agent comprises less than 1% free glycans.

Embodiment 17. The method of embodiment 13, wherein the agent that selectively increases the gut population of one or more bacterial species in (1) comprises one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

Embodiment 18. A method of treating or preventing inflammatory bowel disease (IBD) or risk of IBD in a canine in need thereof comprising administering to the canine an effective amount of
(1) a composition comprising an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(2) a composition comprising an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or
(3) a combination of compositions of (1) or (2),
wherein a sample isolated from the canine in need thereof has levels of a measured bacterial species in step (i) are lower than a pre-determined threshold level of the corresponding bacterial species, or when the levels of the measured species in step (ii) are higher than a pre-determined threshold level of the corresponding bacterial species, and wherein the measured bacterial species comprise one or more of:
(i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis.

Embodiment 19. The method of embodiment 18, wherein the agent that selectively increases the gut population of one or more bacterial species in (1) or that selectively decreases the gut population of one or more bacterial species in (2) comprises mucins.

Embodiment 20. The method of embodiment 18 or 19, wherein the agent comprises porcine mucins obtained from porcine gastrointestinal tract.

Embodiment 21. The method of any one of embodiments 18-20, wherein the agent comprises less than 1% free glycans.

Embodiment 22. The method of any one of embodiments 5-21, wherein the canine is a dog, optionally a domesticated dog.

Embodiment 23. The probiotic composition of any one of embodiments 1-4, for use in a method of any one of embodiments 5-22.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

What is claimed is:

1. A probiotic composition comprising at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques or Slackia faecicanis, and a pharmaceutically acceptable carrier.

2. The probiotic composition of claim 1, further comprising at least one bacterial species selected from Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis or Romboutsia lituseburensis.

3. The probiotic composition of claim 1, further comprising one or both of bacterial species Blautia hansenii and Clostridium perfringens.

4. The probiotic composition of claim 1, further comprising at least one prebiotic selected from: fructo-oligosaccharides, short chain fructo-oligosaccharides, inulin, isomalto-oligosaccharides, pectins, galacto-oligosaccharides, arabinogalactan, xylo-oligosaccharides, chitosan-oligosaccharides, glucomannan, beta-glucans, Konjac, guar, arabic, xanthan gums, modified and resistant starches, polydextrose, or D-tagatose.

5. A method for identifying inflammatory bowel disease (IBD) or risk of IBD in a canine comprising:
(a) measuring the levels of:
(i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis,
in a biological sample from the canine relative to pre-determined threshold expression level of the corresponding bacterial species;
and
(b) determining that the canine has IBD or at risk of IBD when the level of the bacterial species measured in step (a) (i) are lower than a pre-determined threshold level of the corresponding bacterial species, or when the level of the bacterial species measured in step (a) (ii) is higher than a pre-determined threshold level of the corresponding bacterial species.

6. The method of claim 5, wherein the biological sample is a fecal sample.

7. A method for treating inflammatory bowel disease (IBD) or risk of IBD in a canine comprising:
(a) receiving information as to the levels of
(i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis,
in a biological sample from the canine relative to a pre-determined threshold level of the corresponding bacterial species;
(b) determining that the canine has IBD or at risk of IBD when the level of the bacterial species measured in step (a) (i) is lower than a pre-determined threshold level of the corresponding bacterial species, or when the level of the bacterial species measured in step (a) (ii) is higher than a pre-determined threshold level of the corresponding bacterial species; and
(c) treating the canine determined to have IBD or at risk of IBD in step (b), by administering to the canine a therapeutically effective amount of:
(1) a composition comprising an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(2) a composition comprising an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or
(3) a combination of compositions of (1) or (2).

8. The method of claim 7, wherein the biological sample is a fecal sample.

9. The method of claim 7, wherein the agent that selectively increases the gut population of one or more bacterial species in (1) or that selectively decreases the gut population of one or more bacterial species in (2) comprises mucins.

10. The method of claim 7, wherein the agent comprises porcine mucins obtained from porcine gastrointestinal tract.

11. The method of claim 7, wherein the agent comprises less than 1% free glycans.

12. The method of claim 7, wherein the agent that selectively increases the gut population of one or more bacterial species in (1) comprises one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

13. A method for preventing inflammatory bowel disease (IBD) in a canine, comprising administering to the canine an effective amount of
(1) a composition comprising an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(2) a composition comprising an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or
(3) a combination of compositions of (1) or (2).

14. The method of claim 13, wherein the agent that selectively increases the gut population of one or more bacterial species in (1) or that selectively decreases the gut population of one or more bacterial species in (2) comprises mucins.

15. The method of claim 13, wherein the agent comprises porcine mucins obtained from porcine gastrointestinal tract.

16. The method of claim 13, wherein the agent comprises less than 1% free glycans.

17. The method of claim 13, wherein the agent that selectively increases the gut population of one or more bacterial species in (1) comprises one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens.

18. A method of treating or preventing inflammatory bowel disease (IBD) or risk of IBD in a canine in need thereof comprising administering to the canine an effective amount of
(1) a composition comprising an agent that selectively increases the gut population of one or more bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(2) a composition comprising an agent that selectively decreases the gut population of one or more bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis, or
(3) a combination of compositions of (1) or (2),
wherein a sample isolated from the canine in need thereof has levels of a measured bacterial species in step (i) are lower than a pre-determined threshold level of the corresponding bacterial species, or when the levels of the measured species in step (ii) are higher than a pre-determined threshold level of the corresponding bacterial species, and wherein the measured bacterial species comprise one or more of:
(i) at least three bacterial species selected from Bacteroides plebeius, Clostridium saudiense, Fusobacterium varium, Holdemanella biformis, Phascolarctobacterium succinatutens, Prevotella copri, Romboutsia timonensis, Ruminococcus torques, Slackia faecicanis, Blautia acetigignens, Blautia glucerasea, Blautia hansenii, Blautia obeum, Clostridium bartlettii, Clostridium disporicum, Clostridium hiranonis, Clostridium paraputrificum, Clostridium perfringens, Fournierella massiliensis, Fusobacterium mortiferum, Gallintestinimicrobium propionicum, Romboutsia ilealis, Romboutsia lituseburensis, Blautia hansenii or Clostridium perfringens, or
(ii) at least one bacterial species selected from Bacillus bogoriensis, Butyricicoccus pullicaecorum, Campylobacter upsaliensis, Clostridium hylemonae, Clostridium saccharolyticum, Collinsella intestinalis, Collinsella stercoris, Escherichia coli, Faecalimonas umbilicate, Gluceribacter canis, Hespellia stercorisuis, Lachnoclostridium pacaense, Lachnoclostridium phocaeense, Laedolimicola ammoniilytica, Mediterraneibacter glycyrrhizinilyticus, Megamonas funiformis, Phocaeicola faecicola, Romboutsia hominis, Ruminococcus gnavus, Sutterella massiliensis or Tyzzerella nexilis.

19. The method of claim 18, wherein the agent that selectively increases the gut population of one or more bacterial species in (1) or that selectively decreases the gut population of one or more bacterial species in (2) comprises mucins, and wherein the canine is a domesticated dog.

20. The method of claim 18, wherein the agent comprises porcine mucins obtained from porcine gastrointestinal tract, and wherein the agent comprises less than 1% free glycans.
